SEQUENCE LISTING (2 sequences)

SEQ ID NO: 1 - DNA, 20 bp, Artificial Sequence
  RNF213 mutation_F
gctgcatcacaggaaatgac

SEQ ID NO: 2 - DNA, 20 bp, Artificial Sequence
  RNF213 mutation_R
aaggagtgagccgagtttga

(19) United States
(12) Patent Application Publication
(10) Pub. No.: US 2018/0030535 A1
(43) Pub. Date: Feb. 1, 2018

(54) BIOMARKER FOR DIAGNOSIS OF MOYAMOYA DISEASE

(71) Applicant: SAMSUNG LIFE PUBLIC WELFARE FOUNDATION, Seoul (KR)

(72) Inventors: Oh Young BANG, Seoul (KR); Gyeong Joon MOON, Gyeonggi-do (KR); Jong-Won CHUNG, Seoul (KR); Mi Jeong OH, Seoul (KR); Dong Hee KIM, Seoul (KR); Soo Yoon KIM, Gyeonggi-do (KR); Yeon Hee CHO, Seoul (KR)

(73) Assignee: SAMSUNG LIFE PUBLIC WELFARE FOUNDATION

(21) Appl. No.: 15/658,563

(22) Filed: Jul. 25, 2017

(30) Foreign Application Priority Data
Jul. 26, 2016 (KR) 10-2016-0094644
Jun. 28, 2017 (KR) 10-2017-0081683

(51) Int. Cl. C12Q 1/68; G01N 33/50; C07K 16/28; G01N 33/68

(52) U.S. Cl. CPC C12Q 1/6883; G01N 33/6893; G01N 33/5088; C07K 16/28; C12Q 2600/156; G01N 2800/32; G01N 2333/705

(57) ABSTRACT

Provided are a biomarker composition for the diagnosis of moyamoya disease, including caveolin-1, a kit for the diagnosis of moyamoya disease, a method of providing information for moyamoya disease diagnosis, and a method of screening a moyamoya disease therapeutic agent. According to the present disclosure, specific treatment for moyamoya disease is possible by distinguishing the disease from atherosclerosis, which is a similar cerebrovascular stenosis disease, and thus unnecessary factors or risk factors that may be accompanied by side effects may be avoided and, therefore, may be applied to effective MMD therapies.

### CROSS-REFERENCE TO RELATED APPLICATION

This application claims priority to and the benefit of Korean Patent Applications No. 10-2016-0094644, filed on Jul. 26, 2016, and 10-2017-0081683, filed on Jun. 28, 2017, the disclosure of which is incorporated herein by reference in its entirety.

### FIELD

The present disclosure relates to a biomarker composition for the diagnosis of moyamoya disease which includes caveolin-1, a kit for the diagnosis of moyamoya disease, a method of providing information for moyamoya disease diagnosis, and a method of screening therapeutic agent.

### BACKGROUND

Moyamoya disease is a cerebrovascular disorder of unknown origin, and is characterized in that, through a cerebral angiogram, progressive stenosis of the distal bilateral internal carotid artery and main branches (the middle cerebral artery and the anterior cerebral artery) that enter the cerebrum and a blood vessel network (basilar collateral circulation blood vessels; Moyamoya blood vessels), in which abnormal fine blood vessels grow from the base of the brain, which is a neighboring part of the narrowed artery, and gather, are shown (see FIG. 1). The moyamoya disease is a relatively rare disease that occurs in the Far East Asia including Korea, China, Japan, and the like, and occurs in children in the form of repeated transient ischemic attacks and commonly occurs in adults as cerebral hemorrhage.

With regards to the diagnosis of moyamoya disease, the cause of the disease has not yet been found, and thus, invasive angiography is currently the only way to diagnosis the disease. However, characteristic moyamoya vessels are specific angiographic findings, but are not observed in all patients. In particular, unlike children with moyamoya disease, there are common cases in which these moyamoya vessels are not observed in adults with moyamoya disease, and thus, in many cases, it is difficult to distinguish the moyamoya disease from stenosis due to atherosclerosis. In addition, there are many cases in which moyamoya vessels are not observed at an early stage according to progression of the disease, and thus sequential angiography may be required.

It is very important in treatment of patients to diagnose moyamoya disease by distinguishing it from other diseases that cause angiostenosis such as atherosclerosis. That is, this is because, in the case of atherosclerosis, the progression of stenosis or the onset of cerebral infarction is effectively prevented using statin as an antiplatelet agent and stent insertion is helpful, while, in the case of moyamoya disease, drug treatment is not helpful for the amelioration of the disease, stent blockage mostly occurs even after stent insertion. In contrast, a bypass surgery currently known as moyamoya disease treatment is not recommended for patients with blockage due to atherosclerosis.

Meanwhile, caveolae are known as being 50 to 100 nm flask-type grooves that play a vital role in endocytosis, being abundant in endothelial cells, and playing an important role in vesicular trafficking and signal transduction of endothelial cells (see FIG. 2).

In this regard, it has been reported that caveolin-1, which is a membrane protein essential for forming/maintaining caveolae, not only simply functions as a caveolae scaffolding protein, but also plays an important role in mobilizing vascular progenitor cells from bone marrow, and is involved in the onset of diseases such as tumors and the like (Frank P G et al., Arterioscler Thromb Vasc Biol 2003; 23:1161-8), and the relevance of caveolin-1 to moyamoya disease has not yet been reported.

### SUMMARY

Therefore, the inventors of the present disclosure verified the fact that the level of caveolin-1 specifically decreased in blood samples of moyamoya disease patients by comparing with normal individuals and atherosclerosis patients, thus completing the present disclosure.

Accordingly, the present disclosure provides a biomarker composition for the diagnosis of moyamoya disease which includes caveolin-1, a kit for the diagnosis of moyamoya disease, a method of providing information for moyamoya disease diagnosis, and a method of screening therapeutic agent.

Additional aspects will be set forth in part in the description which follows and, in part, will be apparent from the description, or may be learned by practice of the presented embodiments.

According to an aspect of the present disclosure, a biomarker composition for the diagnosis of moyamoya disease includes caveolin-1.

In one embodiment, a level of caveolin-1 mRNA or protein is decreased in an individual with moyamoya disease.

According to another aspect of the present disclosure, a composition for the diagnosis of moyamoya disease includes a formulation for measuring an expression level of caveolin-1 mRNA or protein.

In one embodiment, the formulation for measuring an expression level of caveolin-1 mRNA includes an antisense oligonucleotide, a primer, or a probe, specifically binding to a caveolin-1 gene.

In another embodiment, the formulation for measuring an expression level of caveolin-1 protein includes an antibody specifically binding to caveolin-1 protein.

According to another aspect of the present disclosure, a kit for the diagnosis of moyamoya disease includes the composition described above.

In one embodiment, the kit is a DNA microarray chip or a protein chip.

According to another aspect of the present disclosure, a method of providing information for moyamoya disease diagnosis includes measuring an expression level of caveolin-1 mRNA or protein from a sample of an individual.

In one embodiment, the method further includes determining that, as a result of measurement of the expression level of caveolin-1, in a case in which the expression level is decreased compared to a normal control, the case is diagnosed as moyamoya disease.

In another embodiment, the measuring is performed by one selected from reverse transcription-polymerase chain reaction (RT-PCR), competitive RT-PCR, real-time RT-PCR, RNase protection assay (RPA), and Northern blotting.

In another embodiment, the measuring is performed using an antigen-antibody reaction.

In another embodiment, the antigen-antibody reaction is performed by one selected from enzyme linked immunosorbent assay (ELISA), western blotting, radioimmunoassay (RIA), radioimmunodiffusion, Ouchterlony immunodiffusion, rocket immunoelectrophoresis, immunohistostaining, immunoprecipitation assay, complement fixation assay, fluorescence activated cell sorting (FACS), and microarray analysis.

In another embodiment, the sample includes one selected from blood, serum, plasma, saliva, lymph, and urine.

According to another aspect of the present disclosure, a method of screening a moyamoya disease therapeutic agent includes: treating a sample derived from an individual with a candidate drug; and detecting an expression profile of caveolin-1.

In one embodiment, the sample includes one selected from blood, serum, plasma, saliva, lymph, and urine.

### BRIEF DESCRIPTION OF THE DRAWINGS

The above and other objects, features and advantages of the present disclosure will become more apparent to those of ordinary skill in the art by describing in detail exemplary embodiments thereof with reference to the accompanying drawings, in which:

FIG. 1 illustrates cerebrovascular angiographic images of a normal individual and a patient with moyamoya disease;

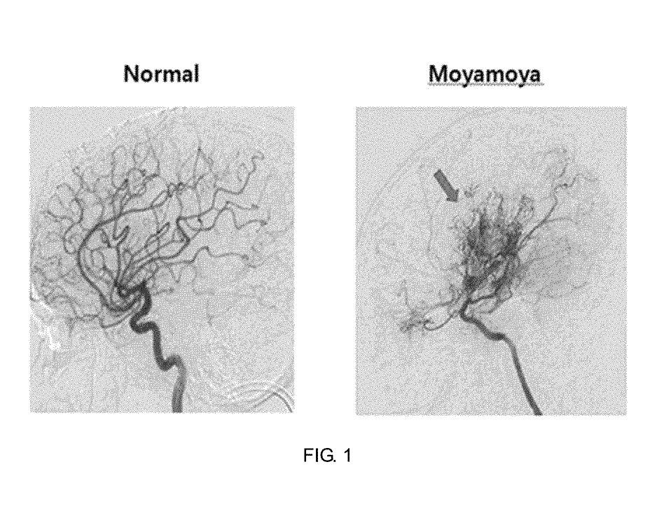

FIG. 2 illustrates an image and a view illustrating caveolae and caveolin-1;

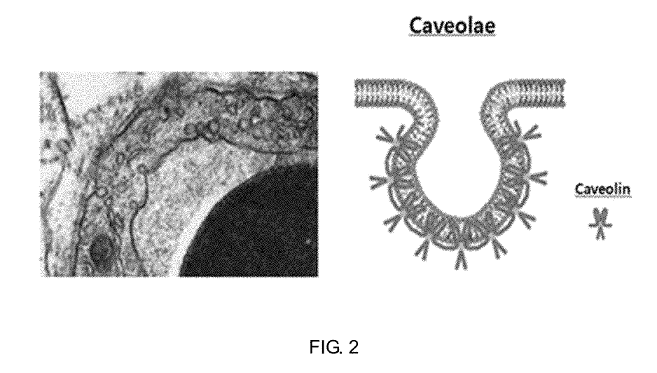

FIG. 3 shows results illustrating a decreased caveolin-1 serum level in patients with moyamoya disease (MMD) verified as an RNF213 mutant, compared to a control and a patient group with intracranial atherosclerotic stroke (ICAS);

FIG. 4 is a receiver operating characteristic (ROC) curve showing that caveolin-1 is effective as a biomarker in moyamoya disease patients diagnosed by angiography;

FIG. 5 is an ROC curve showing that caveolin-1 is effective as a biomarker in genetically diagnosed moyamoya patients;

FIG. 6A is a diagram illustrating path analysis results of the relation between caveolin-1 and moyamoya disease-related genotypes and angiogenesis-related growth factors in the onset of moyamoya disease;

FIG. 6B is a table showing results of FIG. 6A;

FIG. 7 illustrates observation results of the mobility of cells (vascular endothelial cells and smooth muscle cells) constituting blood vessels in which the expression of caveolin-1 was partially suppressed;

FIG. 8 illustrates test results of angiogenesis ability in vascular endothelial cells in which the expression of caveolin-1 was partially suppressed; and

FIG. 9 illustrates test results of changes of vessel maturation-related factors (PDGF and a PDGF receptor) in cells (vascular endothelial cells and smooth muscle cells) constituting blood vessels in which the expression of caveolin-1 was partially suppressed.

### DETAILED DESCRIPTION

Exemplary embodiments of the present disclosure will be described in detail below with reference to the accompanying drawings. While the present disclosure is shown and described in connection with exemplary embodiments thereof, it will be apparent to those skilled in the art that various modifications can be made without departing from the spirit and scope of the invention.

The present invention provides a technique for diagnosing moyamoya disease using caveolin-1 as a biomarker, based on the findings that caveolin-1 specifically decreases in samples of moyamoya disease (MMD) patients.

In the present disclosure, serum samples are collected from MMD patients, intracranial atherosclerotic stroke (ICAS) patients, and a control, expression levels of various proteins thereof are measured and comparatively evaluated, thereby selecting caveolin-1, which is a unique specific biomarker of moyamoya disease which is distinct from atherosclerosis.

In addition, since some conventional MMD patients exhibit family history (genetic history), the correlation between the MMD and a genetic predisposition is evaluated by investigating the relationship between a mutant of Ring finger 213 (RNF213) (GenBank accession number: NM_001256071.1), which is known as a gene related to moyamoya disease, and the level of caveolin-1.

In addition, to evaluate whether a change in caveolin-1 level mediates actions of angiogenesis factors (vascular endothelial growth factor (VEGF), VEGFR2, and endostatin) and endothelial dysfunction factors (ADMA, NO, nitrites, and nitrates) in terms of the onset of moyamoya disease, path analysis is performed.

Furthermore, to evaluate whether a change in level of caveolin-1 affects abnormal angiogenesis of moyamoya disease, the expression of caveolin-1 is partially suppressed at a cell level, and then a migration test and a tubing formation test are performed, and the expression of vessel maturation-related factors (PDGF and a PDGF receptor) is investigated.

In the present disclosure, sequence information of caveolin-1 protein may be acquired from a known database. For example, amino acid sequences of human caveolin-1 protein may be confirmed from Genbank accession number: Q03135. Such caveolin-1 exhibits specific/significant expression changes in samples of a group of moyamoya disease patients, and thus may be used as a diagnostic marker for diagnosing moyamoya disease.

The term “diagnosing” as used herein refers to identifying the presence or characteristics of pathological conditions. In terms of technical goals of the present disclosure, diagnosing means identifying whether moyamoya disease occurs, or identifying whether the disease progresses or is aggravated.

The term “marker” as used herein refers to a material capable of diagnosing moyamoya disease, including organic biomolecules, such as polypeptides, nucleic acids (e.g., mRNA and the like), lipids, glycolipids, glycoproteins, saccharides (monosaccharides, disaccharides, oligosaccharides, and the like), and the like that are related to moyamoya disease. In terms of technical goals of the present disclosure, the marker is caveolin-1, e.g., caveolin-1 mRNA or protein, the expression of which is significantly decreased in moyamoya disease patients.

The present invention also provides a composition for diagnosing moyamoya disease, including a formulation for measuring an expression level of caveolin-1 mRNA or protein.

In the present disclosure, measurement of the expression level of caveoline-1 mRNA is a process of identifying the presence or absence or an expression degree of mRNA of caveolin-1, which is a gene related to moyamoya disease, in a biological sample to diagnose moyamoya disease, and may be performed by measuring the amount of mRNA. Non-limiting examples of analysis methods for this include reverse transcription-polymerase chain reaction (RT-PCR), competitive RT-PCR, real-time RT-PCR, RNase protection assay (RPA), Northern blotting, and a DNA chip. In the present disclosure, the formulation for measuring the level of mRNA of the gene may be antisense oligonucleotides, primer pairs, or probes.

The term “antisense” as used herein refers to an oligomer with a sequence of nucleotide bases and a backbone between sub-units that allows the antisense oligomer to hybridize with a target sequence in RNA by Watson-Crick base pairing, to form a RNA:oligomer heteroduplex within the target sequence, typically with a mRNA. The oligomer may have exact sequence complementarity to the target sequence or near complementarity. These antisense oligomers may block or inhibit translation of the mRNA, and modify the processing of mRNA to produce a splice variant of the mRNA.

The term “primer” as used herein refers to a short nucleic acid sequence having a free 3′-hydroxyl (OH) group that is capable of forming a base pair with a complementary template and acts as a starting point for template strand duplication. A primer may initiate DNA synthesis in the presence of a reagent for polymerization (i.e., DNA polymerase or reverse transcriptase) in a proper buffer solution at a proper temperature and 4 different nucleoside triphosphates.

The term “probe” as used herein refers to a fragment of a nucleic acid such as RNA, DNA, or the like, which corresponds several to hundreds of bases capable of specifically binding to mRNA. Since a probe is labeled, the presence or absence of specific mRNA may be confirmed. A probe may be prepared in the form of an oligo nucleotide probe, a single-stranded DNA probe, a double-stranded DNA probe, an RNA probe, or the like. Selection and hybridization conditions of a suitable probe may be varied based on those disclosed in the art.

The nucleic acid sequence of caveolin-1 related to moyamoya disease is registered in Genebank with Genebank accession numbers: NC_000007.14, NC_018918.2, and NT_007933.16, and thus one of ordinary skill in the art may design an antisense oligonucleotide, a primer pair, or a probe, specifically amplifying a certain region of this gene based on the aforementioned sequence. The antisense oligonucleotide, the primer pair, or the probe of the present disclosure may be chemically synthesized using a phosphoramidite solid support method, or another well-known method.

The expression “measuring an expression level of protein” as used herein refers to a process of identifying the presence or absence and an expression degree of a protein expressed in a biological sample to diagnose moyamoya disease, and the measuring process may be performed by measuring the amount of protein. Non-limiting examples of analysis methods suitable for this include western blotting, enzyme linked immunosorbent assay (ELISA), radioimmunoassay (RIA), radioimmunodiffusion, Ouchterlony immunodiffusion, rocket immunoelectrophoresis, immunohistostaining, immunoprecipitation assay, complement fixation assay, fluorescence activated cell sorting (FACS), and a protein chip. In the present disclosure, the formulation for measuring an expression level of caveolin-1 protein may be an antibody.

The term “antibody” as used herein is known in the art and refers to a specific protein molecule directed against an antigenic site. In terms of technical goals of the present disclosure, the antibody means an antibody specifically binding to a caveolin-1 marker. The type of the antibody of the present disclosure is not particularly limited, and the antibody may also include any fragment of a polyclonal antibody, a monoclonal antibody, or an antibody having an antigenic binding property. In addition, the antibody of the present disclosure may include specific antibodies such as a chimeric antibody, a humanized antibody, a human antibody, and the like. The antibody used in the present disclosure includes functional fragments of antibody molecules, as well as a complete form having two full-length light chains and two full-length heavy chains. The functional fragments of antibody molecules mean fragments retaining at least an antigen-binding function, and include Fab, F(ab′), F(ab′)₂, Fv, and the like.

The present invention also provides a kit for diagnosing moyamoya disease, including the above-described composition. The kit of the present disclosure may further include a composition including one or more types of different components, a solution, or a device that is suitable for use as a means of quantifying caveolin-1 (a primer, a probe, an antibody, or the like) and for a method of analyzing the same. The kit may be an RT-PCR kit, a microarray chip kit, a DNA chip kit, a protein chip kit, or an ELISA kit, but the present disclosure is not limited thereto.

For example, an RT-PCR kit may include, in addition to primer pairs specific to marker genes, a test tube or other appropriate containers, a reaction buffer, deoxynucleotides (dNTPs), enzymes such as Taq-polymerase and reverse transcriptase, a DNase inhibitor, an RNase inhibitor, DEPC-water, sterilized water, and the like. The ELISA kit may include a substrate, an appropriate buffer solution, a secondary antibody labeled with a chromogenic enzyme or a fluorescence material, a chromogenic substrate, and the like to perform immunological detection of an antigen-antibody complex.

The present invention also provides a method of providing information for moyamoya disease diagnosis, including measuring an expression level of caveolin-1 mRNA or protein from a sample of an individual. In the present disclosure, as a result of measurement of the expression level of caveolin-1 from the sample, the method may further include determining that, in a case in which the expression level is decreased compared to that of a normal control, the case is diagnosed as moyamoya disease.

In the present disclosure, the measuring of an expression level of caveolin-1 mRNA may be performed by RT-PCR, competitive RT-PCR, real-time RT-PCR, RPA, Northern blotting, or a DNA chip, but the present disclosure is not limited thereto.

Non-limiting examples of a method of measuring an expression level of caveolin-1 protein include Western blotting, ELISA, RIA, radioimmunodiffusion, Ouchterlony immunodiffusion, rocket immunoelectrophoresis, immunohistostaining, immunoprecipitation assay, complement fixation assay, FACS, and a protein chip.

The present disclosure also provides a method of screening a therapeutic agent for moyamoya disease, including: (a) treating a sample derived from an individual with a candidate drug; and (b) detecting an expression profile of caveolin-1.

The term “sample” as used herein includes a sample such as tissue, a cell, whole blood, serum, plasma, saliva, sputum, or urine that shows a difference in the level of caveolin-1 due to moyamoya disease, but the present disclosure is not limited thereto. For example, the sample may be a blood, serum or plasma sample.

The term “individual” as used herein refers to a subject, and the individual may be all animals, for example, mammals, for example, humans.

Hereinafter, the present disclosure will be described in further detail with reference to the following examples. However, these examples are provided only for illustrative purposes and are not intended to limit the scope of the present disclosure.

### EXAMPLES

### Example 1: Selection of Biomarker for Moyamoya Disease

1-1. Measurement of Protein Expression Level

To select a unique specific biomarker of moyamoya disease (MMD), distinct from atherosclerosis, serum samples were collected from 139 MMD patients, 61 intracranial atherosclerotic stroke (ICAS) patients, and 68 individuals as a control that were distinguished from one another by angiography, and then expression levels of the following proteins were measured using ELISA kits:

caveoline-1 ELISA kit (catalog #E0214h; EIAab);

ADMA ELISA kit (catalog #K7828; Immundiagnostik);

human sE-selectin ELISA kit (catalog #BEK-2089-2P; Biosensis);

human endostatin ELISA (catalog #DNSTO; R&D Systems); and

human lipoprotein-associated phospholipase A2 (Lp-PLA2) ELISA kit (cat #SEA867Hu; Cloud-Clone Corp.).

At this time, ELISA was performed according to the manufacturers' protocols, and analysis was performed using SpectraMax 340PC384 Microplate Reader and SoftMax® Pro Data Analysis Software (Molecular Devices).

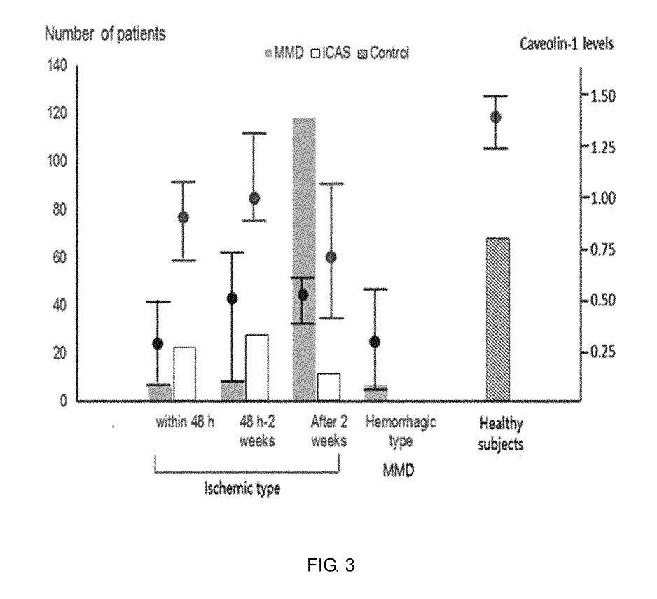

As a result, from the results of FIG. 3, it can be confirmed that the level of caveolin-1 in blood was significantly decreased statistically in MMD patients, and, regardless of the type of moyamoya disease (ischemic or hemorrhagic), such characteristics are maintained regardless of the onset time of the disease.

1-2. Receiver Operating Characteristic (ROC) Curve

An ROC curve is a model for evaluating performance of a particular test, and is a graph representing the correlation between sensitivity and specificity. In the graph, the X axis denotes a false positive rate, the Y axis denotes a true positive rate, and as an ROC curve is drawn closer to the top left, it means higher classification performance.

An area under curve (AUC) is an index representing rationality of a classifier and denotes an area under the ROC curve. The maximum value of the AUC is 1, when a value approximates 1, it means both high sensitivity and high specificity, which indicates excellent classification.

In the ROC curve, the most suitable concentration showing a difference in caveolin-1 level between MMD and ICAS satisfies the conditions: Optimal criterion ≦0.79 (Sensitivity [95% CI], 81.95 [74.4-88.1], Specificity [95% CI], 60.66 [47.3-72.9]).

In particular, the ROC curve was drawn using a STATA version 13.1 program (Stata Corp, College Station, Tex., USA), and the optimal criterion selection, the sensitivity, and the specificity were calculated using a logistic regression analysis technique.

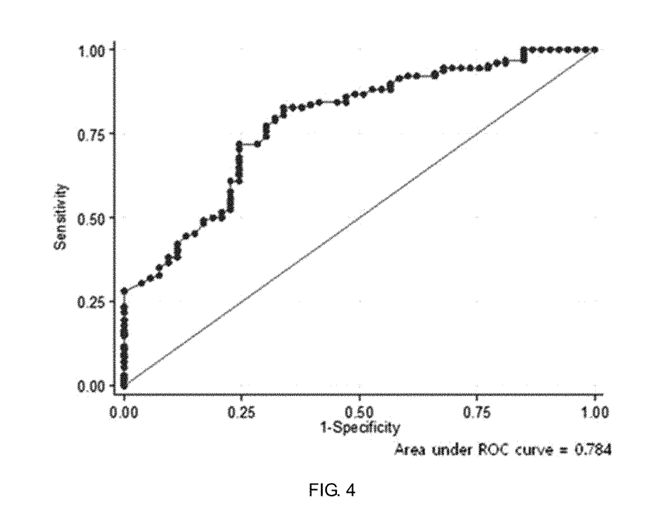

As a result, as illustrated in FIG. 4, it was confirmed that caveolin-1 was effective as a biomarker capable of distinguishing MMD from ICAS, in the case of differential diagnosis by angiography.

### Example 2: MMD-Related Genotype RNF213 Polymorphism Analysis

2-1. Evaluation of Correlation Between RNF213 Variation and Caveolin-1 Level

To identify a difference in the level of caveolin-1 in blood according to a genetic predisposition of MMD, in addition to distinguishing MMD from ICAS, distinguished from each other by angiography, with the level of caverolin-1 in blood, serum samples were collected from ICAS patients and normal individuals, and levels of caveolin-1 therein were identified. That is, a relationship between a group of variants of Ring finger 213 (RNF213) (GenBank accession number: NM_001256071.1) known as a gene related to MMD and the level of caveolin-1 was evaluated.

For this, first, genomic DNA was extracted from peripheral leukocytes using a Wizard genomic DNA purification kit (Promega), and then a c.14429G>A (p.Arg4810Lys) mutation of the RNF213 gene (GenBank accession number: NM_001256071.1) was amplified by PCR (thermal cycler model 9700; Applied Biosystems) using the following primer pair:

Forward primer:
5′-GCTGCATCACAGGAAATGAC-3′
Reverse primer:
5′-AAGGAGTGAGCCGAGTTTGA-3′.

Subsequently, the amplified PCR product was sequenced using a Big-Dye Terminator Cycle Sequencing Ready Reaction kit (Applied Biosystems) on an ABI Prism 3730xl genetic analyzer (Applied Biosystems) to verify RNF213 variants.

As a result of measurement (caveoline-1 ELISA kit; catalog #E0214h, EIAab) of the expression level of caveolin-1 in blood in the verified RNF213 variant group (genetically diagnosed MMD patients), as illustrated in FIG. 3, it was confirmed that the level of caveoline-1 in blood was also significantly decreased in the RNF213 variant group, compared to the control and the ICAS patient group.

2-2. ROC Curve

To evaluate test performance of Example 2-1, an ROC curve was drawn using a logistic regression analysis method (code: Iroc, nograph) of the STATA ver.13.1 program.

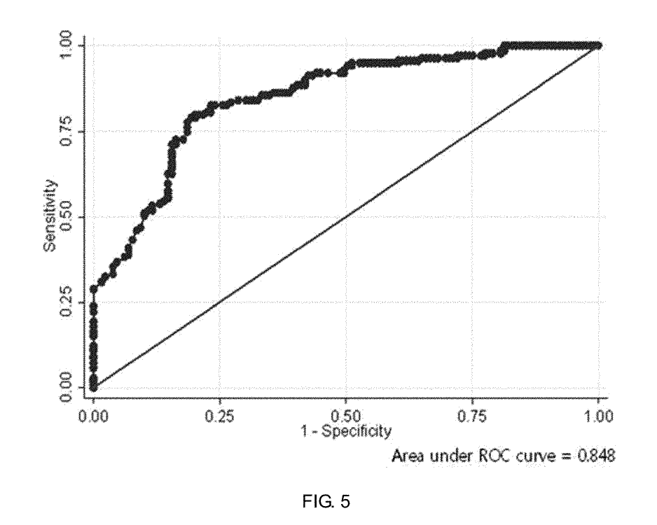

As a result, as illustrated in FIG. 5, it was confirmed that the caveolin-1 biomarker of the present disclosure was effective in differential diagnosis whether individuals have moyamoya disease-related genotypes. These results indicate that caveolin-1 is effective as a biomarker in genetically diagnosed MMD patients.

2-3. Path Analysis

To evaluate whether a change in caveolin-1 level mediates actions of angiogenesis factors (VEGF, VEGFR2, and endostatin) and endothelial dysfunction factors (ADMA, NO, nitrites, and nitrates) in terms of the onset of MMD, path analysis was performed.

That is, the correlation between a genetic marker (RNF213 variation); and a caveolae factor (caveolin-1) as a protein biomarker, angiogenesis factors (VEGF, VEGFR2, and endostatin), and endothelial dysfunction factors (ADMA, NO, nitrites, and nitrates) in MMD patients, ICAS patients, and a control was evaluated.

In particular, path analysis was performed using the STATA ver.13.1 program, and a composite form of two or more regression analyses. Details of the STATA ver.13.1 program using “pathreg” codes are described in http://www.ats.ucla.edu/stat/stata/fag/pathreg.htm.

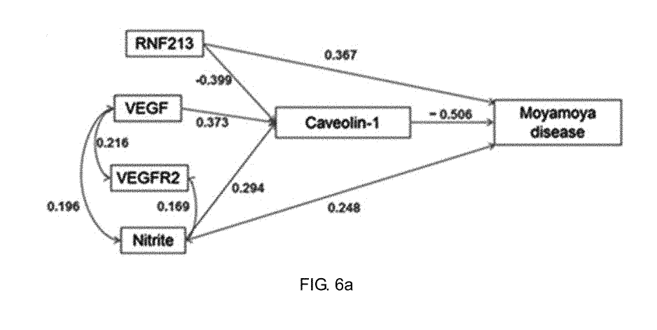

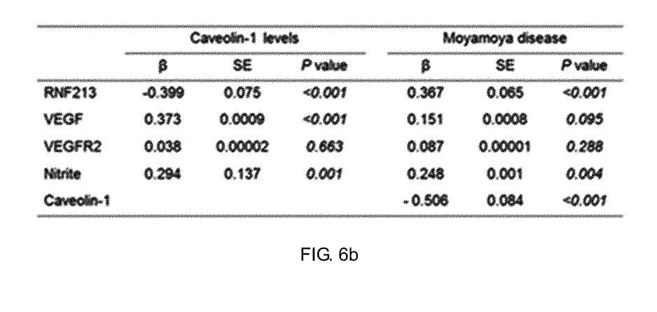

As a result, as illustrated in FIG. 6, it was confirmed that the change in caveolin-1 level was correlated with levels of the MMD genotype (RNF213) and angiogenesis-related growth factors (VEGF) (P value <0.001). In addition, it was confirmed that MMD was simultaneously correlated with the genotype RNF213 and the level of caveolin-1 (P value <0.001).

Thus, according to the present disclosure, it is confirmed that MMD can be diagnosed with high sensitivity/high specificity by measuring the level of caveolin-1 in blood.

### Example 3: Confirmation of Correlation Between Pathological Phenomenon of MMD and Caveolin-1

3-1. Analysis of Change in Mobility of Vascular Cells Due to Decrease in Caveolin-1

To verify the correlation between abnormal angiogenesis, which is a pathological phenomenon of MMD, and a decrease in caveolin-1, caveolin-1 was partially suppressed in a modulation process after transcription in two types of vascular cells (vascular endothelial cells and smooth muscle cells), and mobility thereof was observed.

HUVEC-Primary Umbilical Vein Endothelial Cells (catalog #C2517A; LONZA)

CASMC-Coronary artery SM SmGM-2, cryo amp (catalog #cc-2583; LONZA)

Caveolin-1 siRNA (catalog #sc-29241, Santa Cruz)

At this time, human umbilical vein endothelial cells (HUVECs) were cultured in M199 media containing 20% FBS, 5 U/ml of Heparin, and 3 ng/ml of bFGF, and coronary artery smooth muscle cells (CASMCs) were cultured in Low glucose DMEM media containing 10% FBS. Transfection of caveolin-1 siRNA was performed in HUVECs of passage 5 and CASMCs of passage 7, and the caveolin-1 siRNA was transfected into Lipofectamine according to the manufacturer's protocols of siRNA and cultured in OPTI-MEM media for 6 hours. A control according to the experimental groups was experimented under the same conditions using Control siRNA. 48 hours after siRNA transduction, the centers of plates were horizontally scratched using a pipette tip, and the media were replaced by media containing mitomycin to suppress cell differentiation, and then the samples were observed for 12 hours.

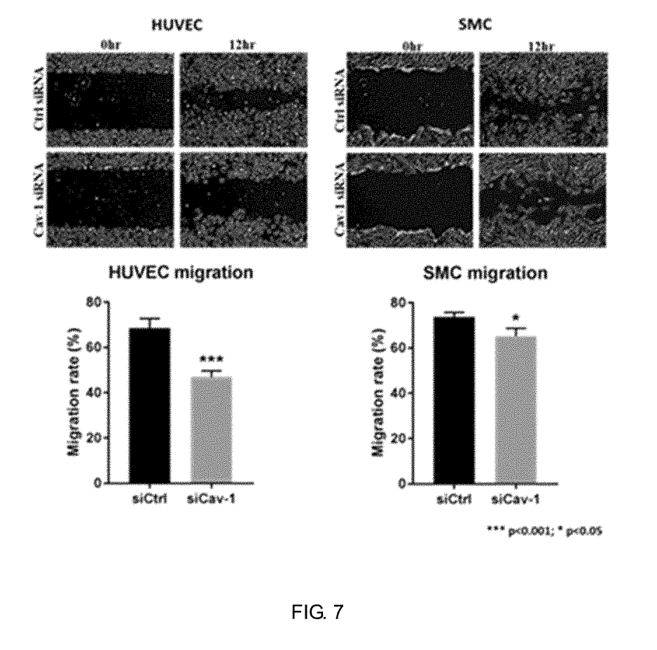

As a result, as illustrated in FIG. 7, it was confirmed that the mobility of cells constituting the two types of blood vessels was significantly decreased due to partial suppression of caveolin-1.

3-2. Analysis of Change in Angiogenesis Ability Due to Decrease in Caveolin-1

To confirm the correlation between abnormal angiogenesis, which is a pathological phenomenon of MMD, and a decrease in caveolin-1, caveolin-1 was partially suppressed in a modulation process after transcription in HUVECs, and mobility thereof was observed.

μ-Slide Angiogenesis (catalog #81506; ibidi)

BD Matrigel™ Matrix Growth Factor Reduced (catalog #356231; BD Bioscience)

Culturing of HUVECs and transfection of siRNA were performed in the same manner as in Example 3-1. 48 hours after siRNA transduction, Matrigel was distributed according to the manufacturer's protocol of μ-Slide, and HUVECs were sub-cultured thereon, and then observed after 8 hours.

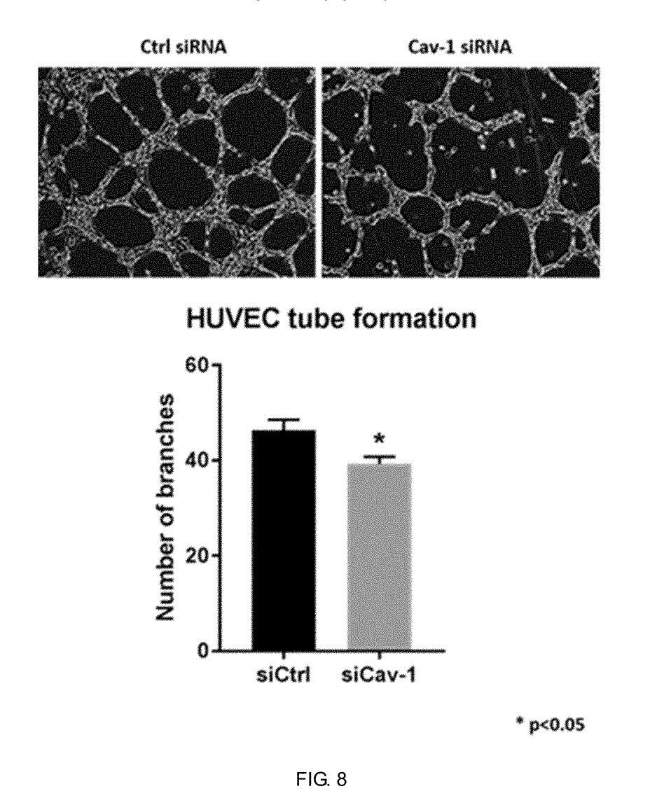

As a result, as illustrated in FIG. 8, it was confirmed that angiogenesis ability of the HUVECs was significantly decreased by partial suppression of caveolin-1.

3-3. Observation of Change in Vessel Maturation-Related Factors Due to Decrease in Caveolin-1

To confirm that the change shown in the cells of Examples 3-1 and 3-2 was based on changes in well-known vessel maturation-related factors, proteins were extracted from the cells and then changes in the factors were observed by Western blot analysis.

Caveolin-1 Antibody (7C8) (catalog #MA3-600; Thermo)

Anti-PDGF-BB Antibody (catalog #LS-C192456; Lifespan Biosciences)

PDGF receptor b (28E1) rabbit mAb (catalog #3169; Cell signaling)

Proteins were classified according to size by sodium dodecyl sulfate (SDS)-polyacrylamide gel electrophoresis using quantified proteins, and then transferred to nitrocellulose membranes, and the amount of each of caveolin-1, PDGF, and PDGF receptor was detected by immunoblotting.

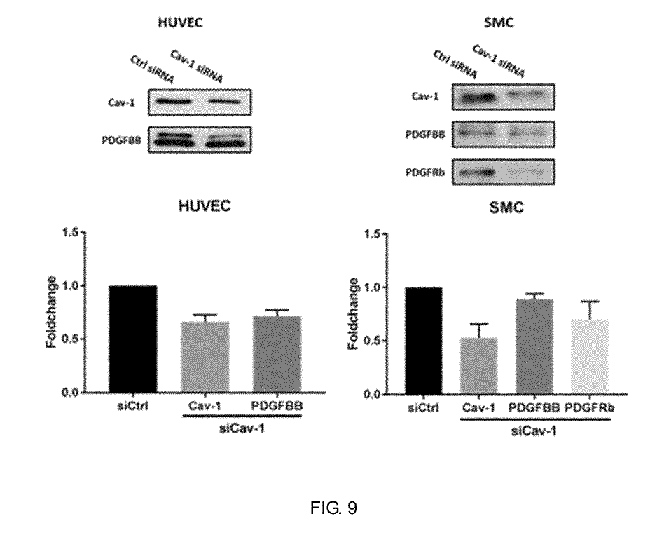

As a result, as illustrated in FIG. 9, it was confirmed that the amount of caveolin-1 was decreased by caveolin-1 siRNA in HUVECs and SMCs, and, accordingly, the amounts of PDGF and the PDGF receptor were decreased.

Thus, from the results of the aforementioned examples, it can be seen that a decrease in the amount of caveolin-1 can be a basis for abnormal angiogenesis, and caveolin-1 can be a MMD biomarker.

As is apparent from the foregoing description, according to the method of diagnosing MMD of the present disclosure, specific treatment for MMD is possible by distinguishing the disease from atherosclerosis, which is a similar cerebrovascular stenosis disease, and thus unnecessary factors or risk factors that may be accompanied by side effects may be avoided and, therefore, can be applied to effective MMD therapies.

In addition, the present disclosure relates to a method of diagnosing MMD by measuring the amount of caveolin-1 specifically changed in blood samples of MMD patients, the method being convenient, noninvasive, and highly accurate.

The foregoing description of the present disclosure is provided for illustrative purposes, and it will be understood by those of ordinary skill in the art to which the present disclosure pertains that the invention may be easily modified in many different forms without departing from the spirit or essential characteristics of the present disclosure. It is therefore to be understood that the above-described embodiments are illustrative in all aspects and not restrictive.

What is claimed is:

1. A method of diagnosing moyamoya disease, the method comprising measuring an expression level of caveolin-1 mRNA or protein from a sample of an individual.

2. The method of claim 1, further comprising determining that, as a result of measurement of the expression level of caveolin-1, in a case in which the expression level is decreased compared to a normal control, the case is diagnosed as moyamoya disease.

3. The method of claim 1, wherein the measuring is performed by one selected from reverse transcription-polymerase chain reaction (RT-PCR), competitive RT-PCR, real-time RT-PCR, RNase protection assay (RPA), and Northern blotting.

4. The method of claim 1, wherein the measuring is performed using an antigen-antibody reaction.

5. The method of claim 4, wherein the antigen-antibody reaction is performed by one selected from enzyme linked immunosorbent assay (ELISA), western blotting, radioimmunoassay (RIA), radioimmunodiffusion, Ouchterlony immunodiffusion, rocket immunoelectrophoresis, immunohistostaining, immunoprecipitation assay, complement fixation assay, fluorescence activated cell sorting (FACS), and microarray analysis.

6. The method of claim 1, wherein the sample comprises one selected from blood, serum, plasma, saliva, lymph, and urine.

7. A method of screening a moyamoya disease therapeutic agent, the method comprising:
treating a sample derived from an individual with a candidate drug; and
detecting an expression profile of caveolin-1.

8. The method of claim 7, wherein the sample comprises one selected from blood, serum, plasma, saliva, lymph, and urine.

9. A kit for the diagnosis of moyamoya disease, the kit comprising a formulation for measuring an expression level of caveolin-1 mRNA or protein.

10. The kit of claim 9, wherein the formulation for measuring an expression level of caveolin-1 mRNA comprises an antisense oligonucleotide, a primer, or a probe, specifically binding to a caveolin-1 gene.

11. The kit of claim 9, wherein the formulation for measuring an expression level of caveolin-1 protein comprises an antibody specifically binding to caveolin-1 protein.

12. The kit of claim 9, wherein the kit is a DNA microarray chip or a protein chip.
